(19) United States
(12) Patent Application Publication
(10) Pub. No.: US 2020/0043585 A1
(43) Pub. Date: Feb. 6, 2020

(54) PATIENT STATUS MONITOR WITH VISUALLY STRONG PATIENT STATUS DISPLAY

(71) Applicant: KONINKLIJKE PHILIPS N.V., EINDHOVEN (NL)

(72) Inventor: Maria Fay HENDRICKSON, Chelmsford, MA (US)

(21) Appl. No.: 16/492,387

(22) PCT Filed: Mar. 8, 2018
(86) PCT No.: PCT/EP2018/055769

§ 371 Date: Sep. 9, 2019

Related U.S. Application Data
(60) Provisional application No. 62/469,549, filed on Mar. 10, 2017.

(51) Int. Cl. G16H 15/00; G16H 10/60

(52) U.S. Cl. CPC G16H 15/00; G16H 10/60

(57) ABSTRACT

A non-transitory storage medium storing instructions readable and executable by at least one electronic processor (56) operatively connected with a display device (60) to perform a patient status monitoring method (100) comprising: retrieving, from at least one database (52), medical information of a patient; computing a state for each of a plurality of body systems or physiologies including at least a neurological body system, a cardiovascular body system, and a respiratory body system by applying clinical decision support (CDS) processing (54) to the retrieved medical information; and displaying, on the display device, a patient status icon (10) with a plurality of icon sections (12, 14 16, 18, 20, 22, 24) corresponding to the plurality of body systems or physiologies, each icon section being displayed with graphical coding that represents the computed state of the corresponding body system or physiology of the patient.

### FIELD

The following relates generally to the patient monitoring arts, patient status monitoring arts, respiratory monitoring arts, cardiovascular monitoring arts, neurological monitoring arts, computer icon generation arts, and related arts.

### BACKGROUND

One of the key responsibilities of clinicians (e.g. nurses, doctors, or so forth treating patients in a hospital or other clinical setting) is to monitor and evaluate a status of the patient. However, this monitoring and evaluation of the patient is challenging, particularly in the case of patients in an intensive care units (ICU), Cardiac Care unit (CCU), or other critical care unit. The clinician in the critical care unit is bombarded with large volumes of patient data from multiple sources. This data comes from devices, from other systems, and from patient assessments. In one study, it was found that on an average 1341 items of clinical data was charted in an adults ICU chart per day (see Manor-Shulman O, Beyene J, Frndova H, Parshuram C S. Quantifying the volume of documented clinical information in critical illness. J Crit. Care 2008; 23(2): 245-250.). The same study found this increased up to 2354 for children receiving certain therapies. This challenge is further expressed in the DIKW pyramid which describes the transformation of data into information into knowledge and then into wisdom (see Henry, Nicholas L. (May-June 1974). “Knowledge Management: A New Concern for Public Administration”. Public Administration Review 34 (3): 189. doi:10.2307/974902. JSTOR 974902). Clinical evaluations and decisions cannot be made effectively or efficiently until data has been transformed into information or knowledge. Often this is done by the clinician themselves, which can lead to delays and/or errors. Some healthcare information systems present the deluge of data in an automated format, but as raw data without substantial post-processing.

Some systems do transform data into information and knowledge, thereby reducing the cognitive load on the clinician and making healthcare decisions more effective. In one approach, the patient status monitoring system displays a “dashboard” for each patient, which includes a row or matrix of body system icons for the patient in a bed. Each body system icon indicates the status of a corresponding body system (e.g. cardiovascular system, respiratory system) with a color. In one conventional color scheme, white indicates normal, yellow indicates abnormal, and red indicates a severe impairment. The indication of patient status using body system icons is an approach often found in clinical information systems. The health status of a patient is suitably determined by applying clinical decision support (CDS) algorithms developed by appropriate medical experts or medical domain-specific medical organizations. The patient status monitor may monitor a large number of patients, e.g. every patient in the critical care unit, using a separate dashboard of body system icons for each patient in the critical care unit.

The following discloses new and improved systems and methods to overcome these problems.

### SUMMARY

In one disclosed aspect, a non-transitory storage medium stores instructions readable and executable by at least one electronic processor operatively connected with a display device to perform a patient status monitoring method comprising: retrieving, from at least one database, medical information of a patient; computing a state for each of a plurality of body systems or physiologies including at least a neurological body system, a cardiovascular body system, and a respiratory body system by applying clinical decision support (CDS) processing to the retrieved medical information; and displaying, on the display device, a patient status icon with a plurality of icon sections corresponding to the plurality of body systems or physiologies, each icon section being displayed with graphical coding that represents the computed state of the corresponding body system or physiology of the patient.

In another disclosed aspect, a patient status monitoring method includes: retrieving, from at least one database, medical information of a patient; computing a state for each of a plurality of body systems or physiologies including at least a neurological body system, a cardiovascular body system, and a respiratory body system by applying clinical decision support (CDS) processing to the retrieved medical information; and displaying, on the display device, a patient status icon with a plurality of icon sections corresponding to the plurality of body systems or physiologies, each icon section being displayed with a color encoding the computed state of the corresponding body system or physiology of the patient, wherein the icon sections corresponding to body systems form a map of the corresponding body systems in a coronal plane of the patient's body.

In another disclosed aspect, a patient status monitoring apparatus includes at least one database containing medical information of the patient. The medical information of the patient includes at least neurological body system information of the patient, cardiovascular body system information of the patient, and respiratory body system information of the patient. A clinical decision system is configured to apply clinical decision support (CDS) processing to compute a state of each of the neurological information, cardiovascular information, and respiratory information of the patient. Each computed state is one of a first state indicating that a status of the patient is normal, a second state indicating that a status of the patient is abnormal but not critical, a third state indicating that a status of the patient is critical, and a fourth state indicating that a status of the patient is unknown. At least one electronic processor is programmed to: generate an icon with a plurality of sections divided by one or more border lines in which the plurality of sections includes at least a section for neurological information comprising an area located on a top portion of the icon; a section for cardiovascular information comprising an area centrally located on the icon; and a section for respiratory information comprising an area centrally located on the icon along the vertical direction and to the sides of the section for cardiovascular information along the horizontal direction; and graphically coding the corresponding sections of the icon based on computed state of the patient of the corresponding body system so that each section exists in one of a first, second, third, or fourth color in which the first color corresponds to the first state, the second color corresponds to the second state and comprising white, the third color corresponds to the third state and comprising a color different from the first and second colors, and the fourth color corresponds to the fourth state and comprising a color other than white different from the first, second, and third colors. A display device is configured to display the icon with each of the sections being in one of the first, second, third, or fourth states.

One advantage resides in providing a patient status monitor with a patient status icon having a strong visual that can used in displays containing small number of patients, or optionally on displays that monitor patient populations.

Another advantage resides in providing a patient status icon that is obscure enough to the general public so that it could be used on white boards and can address concerns patient confidentiality.

Another advantage resides providing in a patient status icon with sections that visually map to the locations of body systems, thereby facilitating rapid comprehension of the constituent body systems or physiologies being represented.

Another advantage resides in providing a patient status icon that provides a strong visual representation for those body systems particularly at risk.

Another advantage resides in providing a patient status icon with various shapes to distinguish different types of patients or patients at different treatment phases.

Another advantage resides in providing a patient status icon with additional visuals indicating therapies being administered to a patient.

Another advantage resides in providing a patient status icon with selectable features to find for more details on that body system corresponding to the selected section.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the invention.

FIG. 1 diagrammatically shows a patient status icon according to one aspect.

FIGS. 2A and 2B diagrammatically show the patient status icon of FIG. 1 with different sections shaded.

FIG. 3 diagrammatically shows a device for measuring respiration of a patient according to one aspect.

FIG. 4 diagrammatically shows an operational flow chart for generation of the icon of FIG. 1.

FIGS. 5A-5D diagrammatically show medical therapy icons on the icon of FIG. 1.

FIGS. 6A-6E diagrammatically show different shapes of the icon of FIG. 1.

FIG. 7 diagrammatically shows a display screen with patient information from which the icon of FIG. 1 can be shown.

FIG. 8 diagrammatically shows a selected patient of FIG. 7 with the corresponding patient status icon of FIG. 1.

### DETAILED DESCRIPTION

The following relates to a new patient status icon, which is designed to be more readily interpreted when displayed on a small scale, such as in the context of dozens of similar icons on a white board or nurses' monitoring station overview screen for a large medical facility. Existing icons employ known symbols representing various body systems (e.g., brain/neurological; cardiac; respiratory; etc.) which may be color coded to represent normal, abnormal, or critical (e.g. white, yellow, red respectively).

Current icon systems have some disadvantages. Some information systems manage the care of intensive care patient populations, often in the hundreds. However, the body system icons are not visually strong enough to identify patients at risk within a population display. The physician reviewing the screen must be able to review across multiple patients and identify those that need special attention. However, the row of body system icons of each patient bed dashboard must be reduced in size to fit a large number of patients on the display. In this reduced size, it can be difficult for the nurse or physician to isolate and recognize a single body system icon of a single patient which is indicating a clinically urgent problem.

Another concern relates to patient privacy. Most care units in the hospital have a white board posted on the wall near the central station listing the patients, their rooms, and important information such as age, gender, and health status. It is a valuable tool for team communication, for example enabling key information to be transferred during nurse shift changes or when a physician visits patients in the care unit. Updating the white board manually is laborious and can be error prone. Leveraging the patient status monitor to display an electronic “white board” in which the status of each patient is represented by the corresponding bed icon would be advantageous. Again, however, existing dashboards of patient status monitors do not provide visually strong representations. A “visually strong” representation should convey the patient information in a concise manner that is easily interpreted without detailed examination (by reading the representation at a glance) and conveys critical information such as indications of body systems or physiologies in critical condition, identification of critical therapies being administered (mechanical ventilation, vasopressor therapy, or so forth), and general patient information such as the status (pre-operative, post-operative, et cetera) in a manner that can be grasped in an instant as the nurse or doctor scans the display.

The disclosed patient icon uses a geometrical shape that maps to the layout of the human body in the coronal plane. The coronal plane is the standard anatomical plane that divides the body into dorsal and ventral (bank and front, or posterior and anterior) portions. The coronal plane is sometimes referred to as the frontal plane. In the coronal plane, the heart (the most critical component of the cardiovascular system) can be viewed as being centrally located, with the head (containing the brain, i.e. the most critical component of the neurological body system) located above the heart, the left and right lungs (the most critical components of the respiratory system) located to opposite sides of the heart. In accord with this mapping of critical organs in the coronal plane, the illustrative patient icon places the cardiac and respiratory regions centrally and larger compared with other regions to emphasize these life-critical body systems. (Making the cardiac and respiratory regions larger may not reflect actual relative organ sizes which can vary amongst individuals, but does reflect the criticality of these life-critical body systems). The resulting icon is more easily read when reduced to small scale, and the layout alignment with the human body improves the intuitiveness of the icon. In some embodiments, the state of one or more physiologies may also be represented, e.g. boundary lines to represent hematology status.

Another optional aspect of the disclosed patient status icon is to represent therapies, preferably using graphical representations of fluid flow or other therapeutic activity (e.g. a constriction shown to illustrate vasopressor therapy). Another optional aspect is the use of a white color to indicate a normal body system contrasted with a background color to represent a system whose status is uncertain. This is useful since knowledge that a system is normal versus unknown can materially affect medical decision-making. There may also be various icon shapes to represent various medical workflow states, e.g. pre-operative versus post-operative, or variants to represent specific medical categories, e.g. an extra central portion representing the status of the fetus in the case of pregnant patient.

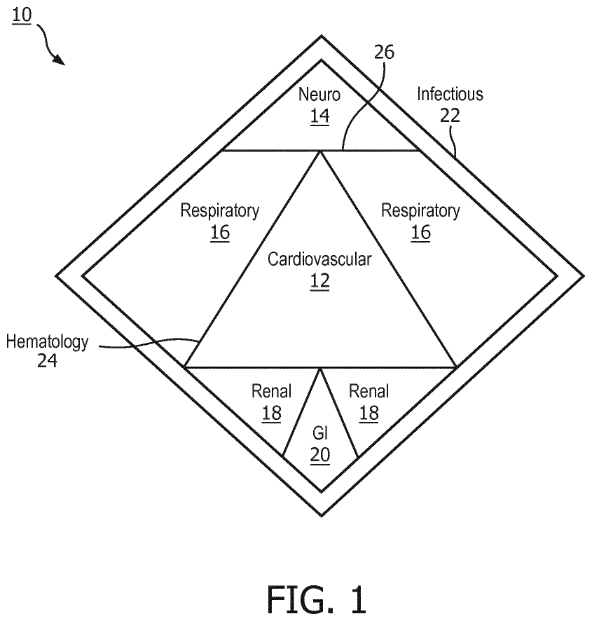

With reference to FIG. 1, an illustrative patient status icon 10 for displaying a status of one or more body systems or physiologies of a patient is shown. As shown in FIG. 1, the patient status icon 10 includes a plurality of sections or areas 12, 14, 16, 18, 20, and 22. Each of these sections correspond to a respective body system or pathology of the patient (e.g., cardiovascular body system, respiratory body system, neurological body system, gastrointestinal (GI) body system, renal body system, hematology physiology, systemic infection physiology, and the like). For example, the patient status icon 10 includes at least a cardiovascular icon section 12, a neurological icon section 14, and a respiratory icon section 16. As shown in FIG. 1, the cardiovascular icon section 12 corresponds to the cardiovascular body system and comprises an area that is centrally located on the icon 10. The neurological icon section 14 corresponds to the neurological body system, and comprises an area that is located above the cardiovascular icon section 12. The respiratory icon section 14 corresponds to the respiratory body system, and includes left and right sub-sections comprising areas which are located left and right, respectively, of the cardiovascular icon section 12.

The illustrative patient status icon 10 also includes a gastrointestinal (GI) icon section 18 corresponding to the gastrointestinal body system. The GI icon section 18 comprises an area located below the cardiovascular icon section 12. A renal icon section 20 corresponding to the renal body system is also located below the cardiovascular icon section 12, i.e. comprises an area located below the cardiovascular section 12. The illustrative GI icon section 18 includes left and right sub-sections comprising areas located left and right, respectively, of the renal icon section 20.

Some sections may be embodied as other than area features of the icon. For example, the illustrative patient status icon 10 further includes a systemic infection icon section 22 corresponding to the systemic infection physiology—the systemic infection icon section 22 comprises an outer border of the patient status icon 10, which graphically captures the physiology of a systemic infection which typically infects a substantial portion, or even all, of the patient's body tissues. As another example, a hematology icon section 24 corresponding to the hematology physiology comprises interior border lines 26 of the patient status icon 10.

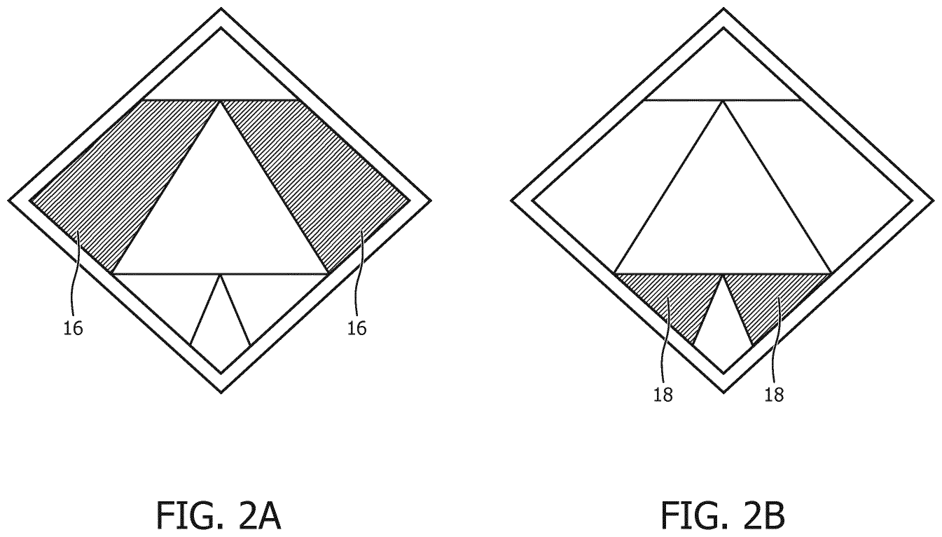

Advantageously, the cardiovascular icon section 12, the neurological icon section 14, and the respiratory icon section 16 are larger in area than the other icon sections 18, 20, 22, and 24 to emphasize these life-critical body systems. As shown in FIGS. 2A and 2B, when any of the icon sections 12, 14, 16, 18, 20, 22, and 24 are filled in with a color representative of a state of the respective body system or physiology (as described in more detail below), the filled-in area of the life-critical icon sections 12, 14, and 16 is more prominent (FIG. 2A) than the filled-in area of the other icon sections 18, 20, 22, and 24 (FIG. 2B). The overall collection of information related to these body systems and physiologies are deemed critical to short-term survival of the patient. For example, if any the icon sections are color-coded to show that a status of the corresponding body system or physiology is critical, then death or permanent impairment the corresponding body system or physiology of the patient is possible.

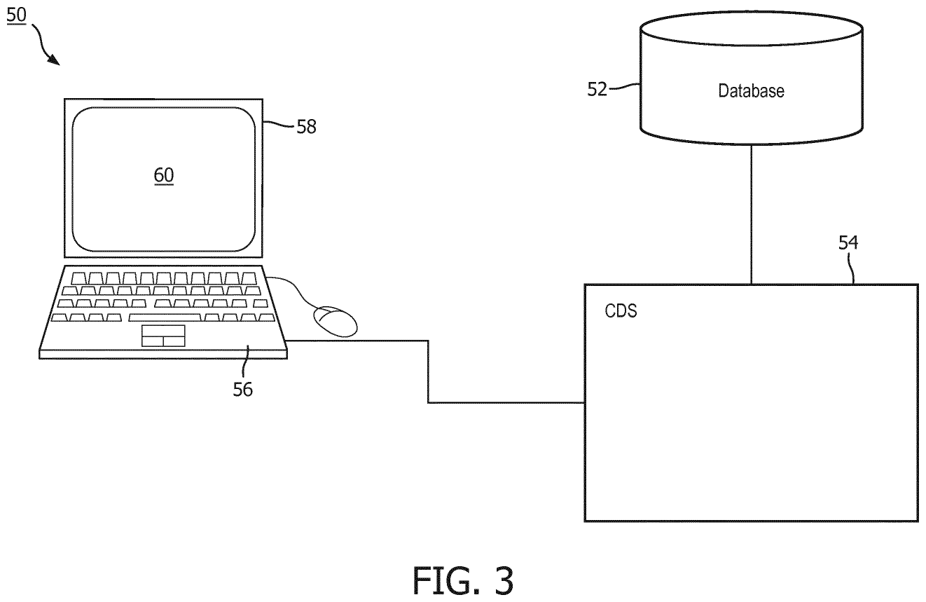

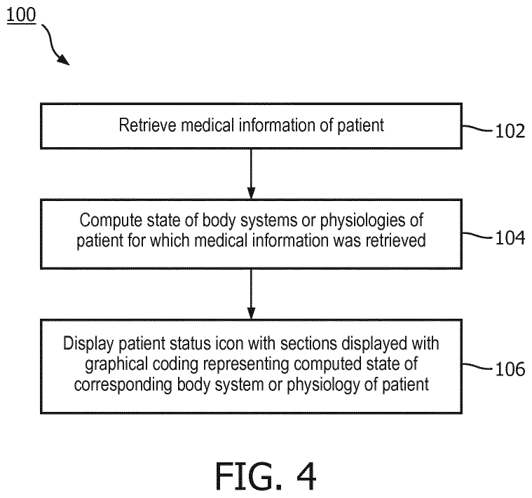

The patient status icon 10 is generated with a patient status monitoring method 100 (FIG. 4) performed by a patient status monitoring apparatus 50 (FIG. 3). Referring first to FIG. 3, the patient status monitoring apparatus 50 includes or has (at least) read access to at least one electronic patient medical database 52, such as an Electronic Medical Record (EMR) database, Electronic Health Record (EHR) database, cardiovascular information system (CVIS), various combinations thereof, or so forth. The database 52 stores and contains medical information of the patient, including at least neurological body system information of the patient, cardiovascular body system information of the patient, and respiratory body system information of the patient. The database 52 may also contain medical information related to systemic infection physiology of the patient, gastrointestinal body system information of the patient, renal body system information of the patient, and hematology physiology information of the patient.

The medical database 52 contains data relevant to assessing the state of various body systems (cardiovascular, respiratory, neurological, et cetera) and physiologies (hematology physiology, systemic infection physiology, et cetera). However, this data is not directly usable to assess the states of those various body systems and physiologies.

To generate body system and physiology state information from the medical data contained in the medical database 52, the patient status monitoring apparatus 50 also includes a clinical decision system (CDS) 54 configured to apply clinical decision support (CDS) processing to compute a state of each of the body systems and physiologies of the patient, as described in more detail below. At least one processor 56 (implemented on, for example, a computer 58) is programmed to generate the patient status icon 10 based on the computed states of the body systems and physiologies. A display device 60 (e.g., of the computer 58) is configured to display the generated patient status icon 10 for use by a medical professional.

With reference to FIG. 4, a patient status monitoring method 100 suitably performed by the patient status monitoring apparatus 50 is described. At 102, medical information of the patient is retrieved from the at least one database 52. At 104, a state for each of a plurality of body systems or physiologies including at least a neurological body system, a cardiovascular body system, and a respiratory body system is computed by applying clinical decision support (CDS) processing with the CDS 54 to the retrieved medical information. At 106, the patient status icon 10 with a plurality of icon sections 12, 14, 16, 18, 20, 22, and 24 corresponding to the plurality of body systems or physiologies is displayed on the display device 60, with each icon section being displayed with graphical coding that represents the computed state of the corresponding body system or physiology of the patient.

At 102, medical information is retrieved from the database 52, which can include not only information of the cardiovascular body system, respiratory body system, neurological body system, but also the gastrointestinal (GI) body system, renal body system, hematology physiology, systemic infection physiology, and the like, of the patient. The particular information retrieved from the database 52 depends on the inputs called for by the CDS processing performed by the CDS 54.

At 104, the CDS 54, using CDS processing, computes a state of each body system or physiology for which information was retrieved from the database 52. For example, the CDS processing can output, for each body system or physiology, a state belonging to a closed set of possible states, e.g. a closed set including at least a first state, a second state, a third state, and a fourth state. In the illustrative example, the first state indicates that a status of the patient represented by the corresponding section 12, 14, 16, 18, 20, 22, and 24 of the icon 10 is normal. The second state indicates that a status of the patient represented by the corresponding section 12, 14, 16, 18, 20, 22, and 24 of the icon 10 is abnormal but not critical (i.e., non-life threatening, or having no or low probability of leading to a permanent debilitation). The third state indicates that a status of the patient represented by the corresponding section 12, 14, 16, 18, 20, 22, and 24 of the icon 10 is critical (i.e., life threatening, or having a significant possibility of leading to permanent debilitation). The fourth state indicates that a status of the patient represented by the corresponding section 12, 14, 16, 18, 20, 22, and 24 of the icon 10 is unknown. This is merely an illustrative system for assigning states to the body systems and physiologies, and other state definitions are contemplated. In general, the number of possible states should be low, e.g. no more than two to four or five states at most, as having an excessive state space makes interpretation of the patient status icon more difficult.

The respective states are computed based on a set of predefined CDS rules developed using clinical information such as may be obtained from studies, and/or guidelines published by medical organizations in their areas of competency (e.g., cardiovascular guidelines published by the American Heart Association or so forth). In one non-limiting illustrative example, for the cardiovascular body system, the following CDS processing may be employed. If the retrieved information indicates that the patient's heart rate is greater than 160 beats per minute or below 40 beats per minute, or if the mean blood pressure of the patient is less than 50 mmHg, then the cardiovascular body system is in a critical (i.e., third) state. In another non-limiting illustrative example, for the respiratory body system, if the respiratory rate is greater than 30 breaths per minute, or less than 5 breaths per minute, or if the oxygen saturation of the patient is less than 85%, then the respiratory body system is in a critical state. In another non-limiting illustrative example, if the respiratory rate is between 25-30 breaths per minute, or between 5-10 breaths per minute, then the respiratory system is in an abnormal (i.e., second) state. In another non-limiting illustrative example, if the patient's body temperature is greater than 38 C, the patient's white blood cell count is greater than 15,000, or the patient's white blood cell count is less than 3,000, then the infectious physiology is in the abnormal (i.e., second) state. In yet another non-limiting illustrative example, if the patient's hematocrit level is less than 19%, or if the patient's hemoglobin level is less than 7 g/dL, then the hematology system of the patient is in the critical (i.e., third) state. These are merely non-limiting illustrative examples, and more generally the CDS 54 preferably implements CDS guidelines embodying the most recent clinical knowledge and expertise for assessing the state of each body system or physiology. In some embodiments, the CDS 54 implements CDS guidelines specific for the class or category of patient being monitored, e.g. employing pediatric CDS guidelines for pediatric patients, guidelines specific for a patient with a certain chronic condition (e.g. a patient with chronic heart failure), or so forth.

The fourth state (indicating the status of the body system or physiology is unknown) is assigned if the CDS rule(s) cannot be executed to determine the state of that body system or physiology due to missing information needed to generate the state. For example, in assessing the state of the systemic infection physiology, the white blood cell count may be needed information; thus, if the white blood cell count is unavailable then the systemic infection physiology is suitably assigned the fourth state indicating that it is not known whether the patient has a systemic infection.

The CDS rules may include numerous features known in the art for providing more accurate and robust assessment of body systems and physiologies. For example, the CDS rules may include rules for selecting or combining measurements of the same physiological parameter. Thus, for example, if the heart rate is available from electrocardiogram (ECG) and pulse oximeter, the CDS rule may select the ECG reading as likely to be more accurate, or alternatively may average the heart rates output by the ECG and pulse oximeter, or may choose one or the other based on a quality metric such as signal-to-noise ratio (SNR) of the ECG and photoplethysmography signals. The CDS rules applied by the CDS 54 are also preferably updated as appropriate to reflect current medical knowledge and practice, e.g. to implement the latest cardiovascular assessment guidelines. (These updates may be manual CDS firmware or software updates or the like).

At 106, once the state of each corresponding body system or physiology of the patient is computed, the icon 10 is generated by the processor 56. The icon 10 is generated so that, when it is displayed on the display device 60, the sections 12, 14, 16, 18, 20, 22, and 24 are displayed with graphical coding comprising a color that represents the computed state of the corresponding body system or physiology of the patient. In an illustrative example, the graphical coding complies with a coding scheme in which white represents a normal computed state of the corresponding body system or physiology (i.e., the first state), yellow represents a non-serious abnormal computed state of the corresponding body system or physiology (i.e., the second state), red represents a serious abnormal computed state of the corresponding body system or physiology (i.e., the third state), and a background color other than white, yellow, or red represents an indeterminate computed state of the corresponding body system or physiology (i.e., the fourth state). When the icon 10 is shown on the display device 60, each of the sections 12, 14, 16, 18, 20, 22, and 24 for which information was retrieved for is displayed with a color representing one of the four states. Based on the respective colors, information about the patient can be determined by a medical professional. While color coding is a preferred graphical coding scheme, other types of graphical coding are contemplated to be employed in the alternative or in addition to color coding. For example, a section that is in the third (most urgent) state may be displayed as a flashing element (cycling on/off), or displayed with color cycling, or displayed using a brighter intensity, so as to emphasize the urgent state of that body system or physiology. By contrast, if the section is in the first (normal) state then it may optionally be displayed at lower intensity to provide further de-emphasis. These are merely illustrative optional features.

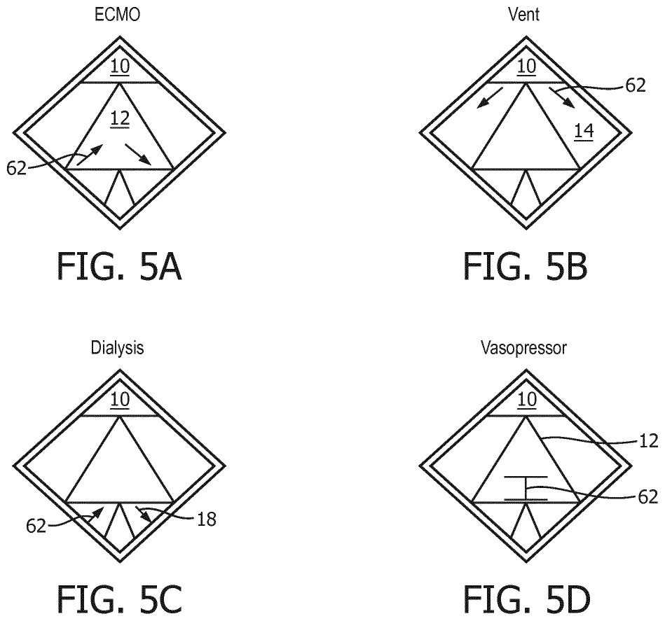

In some examples, during generation of the patient status icon 10, therapy information about the patient can be displayed on the icon. (As used herein, the term “medical information” encompasses information about medical therapies delivered to the patient). For example, from the retrieved patient medical information from the database 52, a medical therapy being delivered to the patient to treat a body system or physiology of the patient undergoing treatment is identified. When a medical therapy is identified, the icon 10 is displayed on the display device 60 and annotating an icon section 12, 14, 16, 18, 20, 22, and 24 corresponding to the body system or physiology undergoing treatment with a medical therapy indicator 62 representing the medical therapy. For example, in FIGS. 5A-5D, several examples of medical therapy indicators 62 are illustrated as arrows representing a fluid flow through the patient produced by the medical therapy. In FIG. 5A, arrows 62 are shown in the cardiovascular icon section 12, and are indicative of extracorporeal membrane oxygenation (ECMO) therapy where blood is removed from the body, oxygenated, and returned to the body. In FIG. 5B, arrows 62 are shown in the respiratory icon section 16 and are indicative of mechanical ventilator providing oxygenated gas to the patient's lungs. In FIG. 5C, arrows 62 are shown in the renal icon section 18 and are indicative of renal dialysis. In FIG. 5D, arrows 62 are shown in the cardiovascular icon section 12 and are indicative of vasopressor therapy. The overall collection of information related to these therapies are deemed critical to short-term survival of the patient, and does not include every therapy being applied to the patient. For example, if any the icon sections are include an indicator 62 to show that the corresponding body system or physiology of the patient is critical, then death or permanent impairment of that corresponding body system or physiology of the patient is possible.

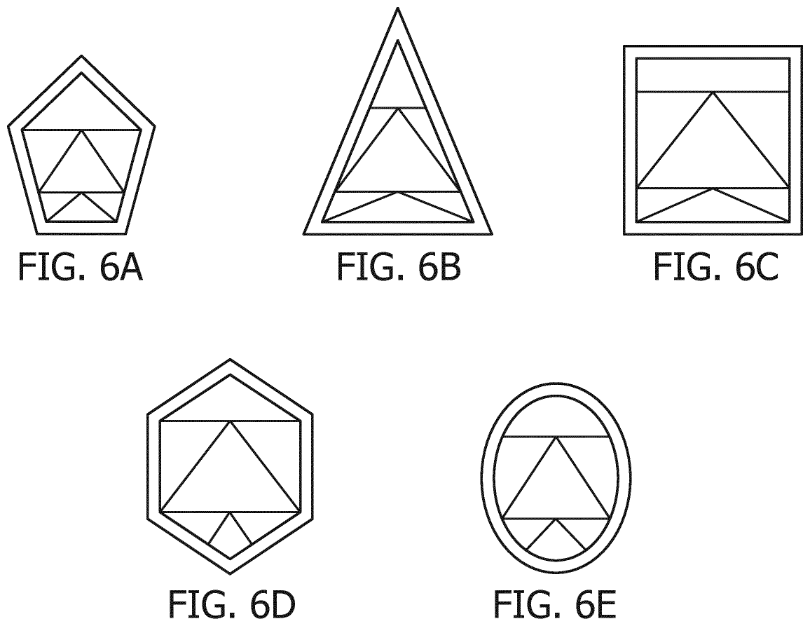

In other examples, a shape of the patient status icon 10 can be changed based on a stage of treatment. As shown in FIG. 1, the icon 10 is shown as a diamond shape. As shown in FIGS. 6A-6E, the patient status icon 10 can have a pentagonal shape, a triangular shape, a square shape, a hexagonal shape, and an elliptical shape. Each of these shapes can be indicative of a stage of treatment of the patient. One of these six shapes can correspond to: (1) a pediatric patient; (2) an adult patient; (3) a pre-operative patient; (4) an operative patient; (5) a post-operative shape; or (6) a women undergoing labor and delivery where the pre-delivery phase is one shape and the post delivery is another gem shape.

As a further optional feature, each section of the patient status icon 10 is optionally user-selectable, e.g. by clicking or double-clicking on the section using a mouse, trackpad, or other pointing-type user input device. As another selection alternative, the display 60 may be a touch-sensitive display, in which case the user can touch the icon section shown in the display 60 to select it. Upon selecting the icon section, context-sensitive information pertaining to the corresponding body system or physiology is brought up in a pop-up window or other display region on the display 60. In similar fashion, selection of one of the medical therapy indicators 62 may bring up a display of context-sensitive information on the therapy (e.g., selecting the mechanical ventilator indicator 62 shown in the example of FIG. 5B may bring up a pop-up window showing current ventilator mode and settings).

The patient status monitor may take various form factors with the data processing operations variously distributed. For example, the patient status monitor may be a nurses' station and/or an electronic white board, with the CDS and icon generation processing being performed locally on the nurses' station monitor processor and/or remotely at a hospital server in communication with the nurses' station via a wired or wireless hospital data network (e.g. wired and/or wireless Ethernet, WiFi, and/or so forth). In another example, a tablet computer or cellular telephone (cellphone) may provide the display component 60, and processing may be performed by an application program (“app”) running on the tablet or cellphone and/or with some processing performed by a hospital server in communication with the tablet computer or cell phone via WiFi, 4G, or another mobile communication network. It will be appreciated that these are not mutually exclusive options, e.g. in one cross-platform approach the patient status icons for all patients in the ICU is generated at a hospital server computer which is then transmitted to both the nurses' station and to each individual nurse's cell phone or other mobile device, where it is displayed. As yet another contemplated variant, if tablets, cellphones or other mobile devices are used as nurses' personal devices, and these devices have GPS, then the display can automatically update to show the patient status icon for the patient (or patients) in the hospital room currently being attended to by the nurse.

### EXAMPLES

A few clinical scenarios using the icons 10 are described. In a first example, the ICU director comes onto the unit. A digital screen representing the white board is displayed by the station. Reviewing the screen, the director observes the patient status icon 10 for each patient, and from those patient status icons readily apprehends that a few patients have some serious issues with multiple systems. The director also notes that he has six post-operative patients in the ICU, based upon the geometrical shapes of the patient status icons. The director talks to the nurse manager and follow-ups with the nurses assigned to the patients. The director also sees a few patients seem to be almost completely normal, thinking those patients might be candidates for transfer out of the ICU. This is discussed with the nurse manager and it is determined that two patients will be transferred out this morning.

In a second example, the ICU nurse is at the bedside of one of their patients. Looking at the clinical information system displayed on the tablet which is carried around, they notice that their other patient cardiovascular system just turned red. Immediately, the nurse clicks on the icons to drill down for the details. Nothing that the patient's blood pressure is lower than before, the nurse goes to the patient's bedside, assesses the patient further, and performs the appropriate action.

In a third example, a physician comes on duty in an electronic ICU (eICU) for the night shift and is receiving the status report from the physician going off-duty. They sit down at the ICU population screen on the electronic white board or nurses' station, which is covering 3 hospitals and 80 ICU patients. The physicians review the screen, and note the patient status icons 10. Noting that 5 patients have multiple systems impacted, the physicians drill down for details to discuss the situation physician coming on duty. After the one physician leaves, the on duty physician calls the nurses at the hospital to discuss the patient, request further assessment, and writes orders as needed to address the patients' condition.

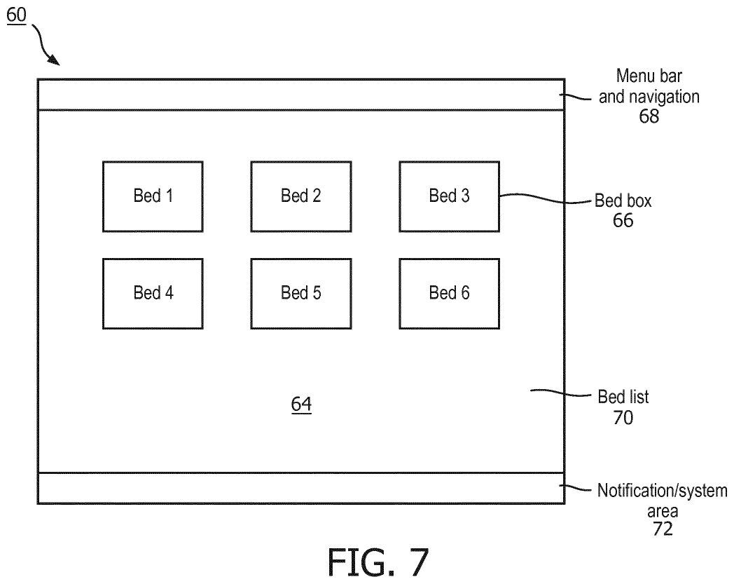

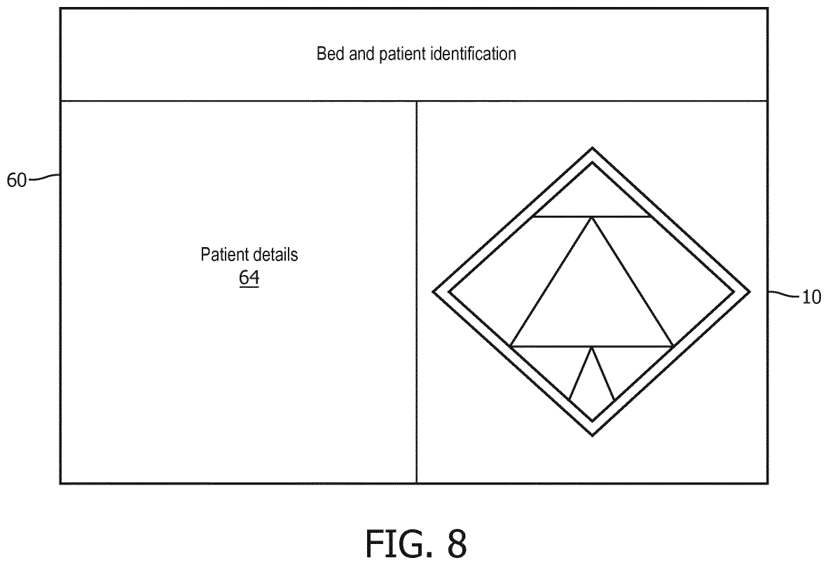

Once generated, the patient status icon 10 is displayed on the display device 60. As shown in FIG. 7, the white board or nurses' station displays other icons related to other patients on the display device 60 which in this example serves as the critical care unit population screen. The patient status icon is part of the clinical information system that includes a population or census screen 64, which includes a set of bed boxes 66 on the census screen on the display device 60. The bed box 66 contains patient information and status for the particular patient. Within the bed box is the patient status icon 10 for that patient. The patient status icon 10 updates real-time as new data comes concerning the patient's status. It can be used by the entire healthcare team. In a usual display on the display device 60, the bed box 66 represents a bed in the care unit, most of these have an assigned patient. When a bed box 66 is selected on the display device 60, the bed box opens up to include its own user interface components, one of which is the patient status icon 10, as shown in FIG. 8. Referring back to FIG. 7, the display also includes a menu bar or navigation bar 68 that contains sub-components describing the name of the product, the user logged in, and other similar information. A bed list 70 contains all of the beds in the care unit as well as the unit name. A notification/system area window 72 includes sub components to display the date/time, notifications, and other information.

It will be appreciated that the illustrative computational, data processing or data interfacing components of the device 10 may be embodied as a non-transitory storage medium storing instructions executable by an electronic processor (e.g., the electronic processor 56) to perform the disclosed operations. The non-transitory storage medium may, for example, comprise a hard disk drive, RAID, or other magnetic storage medium; a solid state drive, flash drive, electronically erasable read-only memory (EEROM) or other electronic memory; an optical disk or other optical storage; various combinations thereof; or so forth.

The population or census screen 64 includes a user interface (UI) that can be written (by way of non-limiting illustrative example) as an Angular JS/HTML5 application that can run on multiple types of devices such as clients, tablets, and others. The patient status icon 10 can be implemented as a component of that product. This framework can include a user interface that is displayed on the various devices. It is written in Angular JS, and includes the template and controller. Web Services which provide the data to the Client UI. Web Services access the database and update. This can be written with any standard programming language such as C#.

Portable components that contain data and are used to communicate data between the UI and the Web services. These may, for example, be represented as JSON objects. These portable components are bound to the UI components in the HTML5 Angular JS. Whenever data is changed in the portable component, the bound UI also changes. A portable component can be made of sub-portable components. For example, the patient portable component may have subcomponents for demographics, laboratory values, etc. In the illustrative UI example, there are various components which are each bound to their own portable component. Each UI component involves a Template which is an html like file and a Controller. The controller collects the POCOs (Plain Old CLR Objects) for the front end UI display and manages the interactions. One controller manages multiple Templates and POCOs such as all of the information displayed in the census screen 64.

The patient status icon 10 is captured in an Angular JS file with a reference to a grouping of SVG (Scalable Vector Graphics) images. SVG are commonly used UI components and support software manipulation of the images. Every body system/concern in the Health Gem is an individual SVG image.

The patient status icon 10 is bound to a subcomponent portable component of a patient portable component. The patient portable component has subcomponents representing each body system and returns as a value the color of the component. The color is based upon clinical algorithms that generate a body system status based upon rules.

The controller who manages the templates obtains the portable component from the Web Services. In this case, the web services returns the patient portable component which has the information about the organ systems and their colors. The web services often use a data provider which accesses the information from the database using stored procedures against something like a SQL database.

The patient status icon 10 is designed to be used by multiple products. This includes the template and the portable component. These two pieces can be used in any product based upon web technologies.

For products which do not employ a web browser interface, the patient status icon 10 SVG can be encapsulated by software code, such as C# to make it a standard Microsoft UI component. As a UI component, it has basic functions to resize, reshape, etc. It also has specialized functions to set the color or status for individual body systems. In this version, a view is displayed to the user as a collection of Microsoft UI components. The view obtains it data and configuration information, called a model, from a backend service. The view could also use the same web services that are used in the web approach.

The disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A non-transitory storage medium storing instructions readable and executable by at least one electronic processor operatively connected with a display device to perform a patient status monitoring method comprising:
retrieving, from at least one database, medical information of a patient;
computing a state for each of a plurality of body systems or physiologies including at least a neurological body system, a cardiovascular body system, and a respiratory body system by applying clinical decision support (CDS) processing to the retrieved medical information; and
displaying, on the display device, a patient status icon with a plurality of icon sections corresponding to the plurality of body systems or physiologies, each icon section being displayed with graphical coding that represents the computed state of the corresponding body system or physiology of the patient.

2. The non-transitory storage medium of claim 1, wherein the plurality of icon sections of the patient status icon includes:
a cardiovascular icon section corresponding to the cardiovascular body system which comprises an area centrally located on the icon;
a neurological icon section corresponding to the neurological body system which comprises an area located above the cardiovascular icon section; and
a respiratory icon section corresponding to the respiratory body system which includes left and right sub-sections comprising areas located left and right, respectively, of the cardiovascular icon section in the patient status icon.

3. The non-transitory storage medium of either one of claim 1, wherein:
the plurality of body systems or physiologies further includes a systemic infection physiology; and
the plurality of icon sections of the patient status icon further includes a systemic infection icon section corresponding to the systemic infection physiology wherein the systemic infection icon section comprises an outer border of the patient status icon.

4. The non-transitory storage medium of claim 1, wherein:
the plurality of body systems or physiologies further includes at least one of (i) a gastrointestinal body system and (ii) a renal body system; and
the plurality of icon sections of the patient status icon further includes at least one of (i) a gastrointestinal icon section corresponding to the gastrointestinal body system and located below the cardiovascular icon section and (ii) a renal icon section corresponding to the renal body system and located below the cardiovascular icon section.

5. The non-transitory storage medium of claim 1, wherein:
the plurality of body systems or physiologies further includes a hematology physiology; and
the plurality of icon sections of the patient status icon further includes a hematology icon section corresponding to the hematology physiology wherein the hematology icon section comprises interior border lines of the patient status icon.

6. The non-transitory storage medium of claim 1, wherein each icon section is displayed on the display device with graphical coding comprising a color that represents the computed state of the corresponding body system or physiology of the patient.

7. The non-transitory storage medium of claim 1, wherein the graphical coding complies with a coding scheme in which white represents a normal computed state of the corresponding body system or physiology, yellow represents a non serious abnormal computed state of the corresponding body system or physiology, and red represents a serious abnormal computed state of the corresponding body system or physiology.

8. The non-transitory storage medium of claim 7, wherein the graphical coding complies with a coding scheme in which a background color other than white, yellow, or red represents an indeterminate computed state of the corresponding body system or physiology.

9. The non-transitory storage medium of claim 1, wherein the patient status monitoring method further comprises:
identifying, from the retrieved medical information, a medical therapy being delivered to the patient to treat a body system or physiology undergoing treatment of the plurality of body systems or physiologies;
wherein the displaying, on the display device, of the patient status icon includes annotating the icon section corresponding to the body system or physiology undergoing treatment with a medical therapy indicator representing the medical therapy.

10. The non-transitory storage medium of claim 9, wherein the medical therapy indicator comprises one or more arrows representing a fluid flow through the patient produced by the medical therapy.

11. The non-transitory storage medium of claim 1, wherein the displaying, on the display device, of the patient status icon includes displaying the patient status icon having a shape indicative of a medical workflow state of the patient, the medical workflow state including at least one of a pre-operative workflow state, an operative state, a post-operative state, an adult patient state, a pediatric patient state, and a pregnancy state indicating a state of both a mother and a fetus.

12. A patient status monitoring method, comprising:
retrieving, from at least one database, medical information of a patient;
computing a state for each of a plurality of body systems or physiologies including at least a neurological body system, a cardiovascular body system, and a respiratory body system by applying clinical decision support (CDS) processing to the retrieved medical information; and
displaying, on the display device, a patient status icon with a plurality of icon sections corresponding to the plurality of body systems or physiologies, each icon section being displayed with a color encoding the computed state of the corresponding body system or physiology of the patient, wherein the icon sections corresponding to body systems form a map of the corresponding body systems in a coronal plane of the patient's body.

13. The method of claim 12, wherein the plurality of body systems or physiologies further includes at least a systemic infection physiology, a gastrointestinal body system, a renal body system, and a hematology physiology, and
wherein the displaying of the patient status icon includes displaying the patient status icon with the plurality of icon sections including:
a cardiovascular icon section corresponding to the cardiovascular body system which comprises a centrally located area of the icon;
a neurological icon section corresponding to the neurological body system which comprises an area located above the cardiovascular icon section;
a respiratory icon section corresponding to the respiratory body system which includes left and right sub-sections comprising areas located left and right, respectively, of the cardiovascular icon section in the patient status icon;
a systemic infection icon section corresponding to the systemic infection physiology which comprises an outer border of the patient status icon;
a gastrointestinal icon section corresponding to the gastrointestinal body system and comprising an area located below the cardiovascular icon section;
a renal icon section corresponding to the renal body system and comprising an area located below the cardiovascular icon section; and
a hematology icon section corresponding to the hematology physiology which comprises interior border lines of the patient status icon.

14. The method of claim 12, wherein the displaying of the patient status icon includes:
color coding each icon section with a color that represents the computed state of the corresponding body system or physiology of the patient, the color coding being in accord with a coding scheme in which:
white represents a normal computed state of the corresponding body system or physiology;
yellow represents a non serious abnormal computed state of the corresponding body system or physiology;
red represents a serious abnormal computed state of the corresponding body system or physiology; and
a background color other than white, yellow, or red representing an indeterminate computed state of the corresponding body system or physiology.

15. The method of claim 12, further including:
identifying, from the retrieved medical information, a medical therapy being delivered to the patient to treat a body system or physiology undergoing treatment of the plurality of body systems or physiologies; and
annotating the icon section corresponding to the body system or physiology undergoing treatment with a medical therapy indicator representing the medical therapy, the medical therapy icon comprises one or more arrows representing a fluid flow through the patient produced by the medical therapy.

16. (canceled)

17. (canceled)

18. (canceled)

19. (canceled)

20. (canceled)
